(19) United States
(12) Patent Application Publication
(10) Pub. No.: US 2025/0268480 A1
(43) Pub. Date: Aug. 28, 2025

(54) APPARATUS FOR DETECTING VITAL PARAMETERS IN AN EAR OF A HUMAN, AND METHOD FOR MEASURING VITAL PARAMETERS OF A HUMAN

(71) Applicants: EURAC RESEARCH, Bolzano (IT); KERR SRL., Bolzano (IT); MINNOVA MED DER MICHAELA EGEBRECHT, Merano (IT)

(72) Inventors: Giacomo STRAPAZZON, Bolzano (IT); Hermann BRUGGER, Bolzano (IT); Michela MASÉ, Bolzano (IT); Alessandro MICARELLI, Bolzano (IT); Andrea STONA, Bolzano (IT); Michaela EGEBRECHT, Merano (IT); Mattia VINANTE, Bolzano (IT); Lijun CHEN, Bolzano (IT)

(21) Appl. No.: 18/858,105

(22) PCT Filed: Apr. 6, 2023
(86) PCT No.: PCT/EP2023/059159

§ 371 Date: Oct. 18, 2024

(30) Foreign Application Priority Data
Apr. 21, 2022 (EP) 22169290.8

(51) Int. Cl. A61B 5/0205; A61B 5/00; A61B 5/024; A61B 5/1455; G01K 13/20

(52) U.S. Cl. CPC A61B 5/02055; A61B 5/14552; A61B 5/6817; G01K 13/223; A61B 5/02416; A61B 5/02438

(57) ABSTRACT

The present invention relates to a device (1) for detecting at least two vital parameters. This device comprises:
an optical sensor device (2) for detecting at least one vital parameter of a living being (4), wherein the optical sensor device (2) comprises a radiation source (6) for emitting radiation (7) and a radiation detector (8) for detecting radiation (7), wherein the radiation source (6) and the radiation detector (8) are arranged next to one another,
an elongated insertion portion (12) for insertion into an ear canal (14) and for positioning the optical sensor device (2) in a portion (16) of the ear canal (14) surrounded by bone,
wherein the optical sensor device (2) is arranged on the elongated insertion portion (12) or is a component thereof and wherein a temperature sensor (18) for detecting the core body temperature is arranged on the elongated insertion portion (12) or is a component thereof,
an evaluation portion (20) coupled to the insertion portion (12), in particular releasably coupled, wherein the evaluation portion (20) and the optical sensor device (2) are at least temporarily, in particular in a coupled state, connected to one another in terms of signaling.

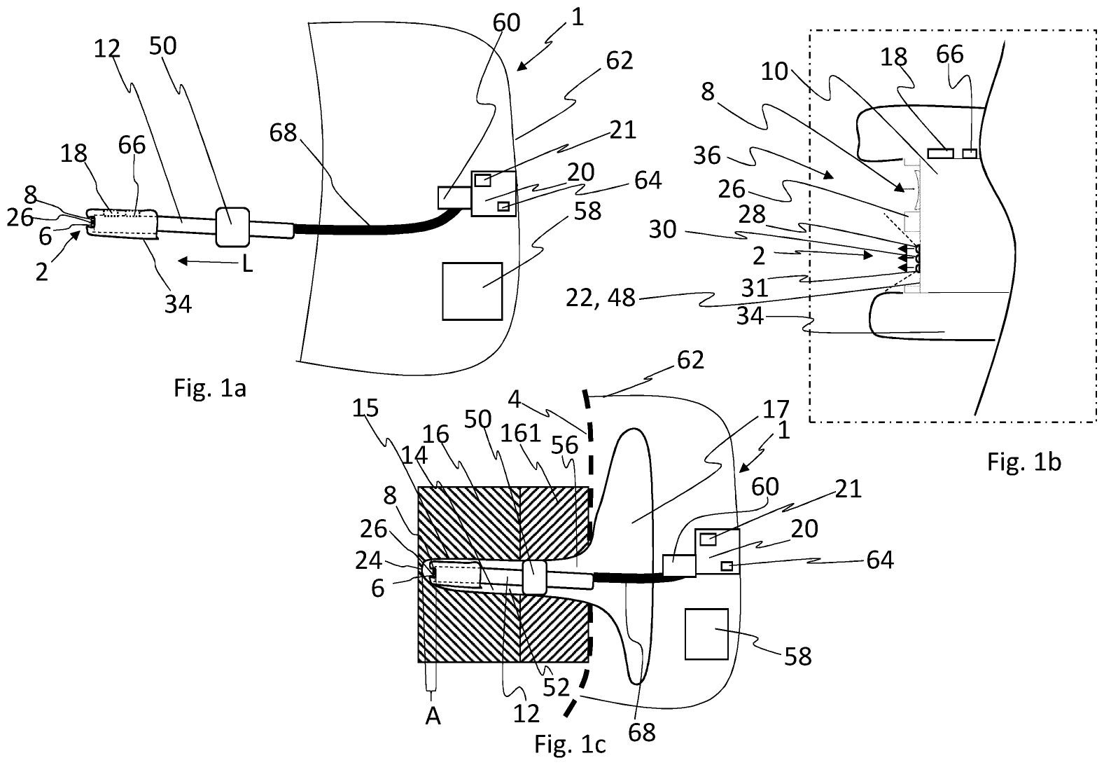

(FIG. 1a)

The present invention relates to a device for detecting vital parameters in a human ear according to claim 1 and to a method for measuring vital parameters of a human according to claim 12.

The publication EP3020327A discloses, for example, an ear thermometer that already delivers favorable results, since a seal of the ear canal to the surrounding area is already proposed in the ear canal, creating a favorable climate in the sealed area. Nevertheless, it is desirable to obtain even more accurate and diverse information about the state of health of a person in distress. Further devices for detecting vital parameters in the ear are disclosed in the documents WO2017/015661A1, WO99/23941A1, DE102017007040A1 and WO2017/203251A1.

It is therefore the object of the present invention to enable a more precise detection of the state of health of a person in distress, in particular a hypothermic person.

The aforementioned object is solved by a device for detecting at least one and preferably two or at least two vital parameters according to claim 1. Such a device preferably comprises at least: an optical sensor device for detecting at least one vital parameter of a living being, wherein the optical sensor device comprises a radiation source for emitting radiation and a radiation detector for detecting radiation, wherein the radiation source and the radiation detector are arranged next to each other, in particular on a PCB. In this context, “side by side” means that the radiation source and the radiation detector are preferably arranged on the same surface and, in particular, in the same orientation. Furthermore, the device preferably comprises an elongated insertion portion for insertion into an ear canal and for positioning the optical sensor device in a portion of the ear canal surrounded by bone. In this context, the term “elongated” means that the extent of the insertion portion in its longitudinal direction is a multiple greater than the extent in a direction orthogonal to the longitudinal direction of the insertion portion, in particular at least three times as long, at least five times as long, at least ten times as long, or, for example, up to twenty times as long or up to fifty times as long. The optical sensor device is particularly preferably arranged on the elongated insertion portion or is a component thereof, and wherein a temperature sensor for detecting the core body temperature is arranged on the elongated insertion portion or is a component thereof. Furthermore, an evaluation portion coupled to the insertion portion, in particular releasably coupled, is provided, wherein the evaluation portion and the optical sensor device are at least temporarily, in particular in a coupled state, connected to one another in terms of signaling.

This solution is advantageous because the arrangement of the radiation source next to the radiation detector enables a very compact design, whereby the optical sensor device can be positioned very deep in the ear canal. Positioning in the area of the tympanic membrane comprises the advantage that the vital parameters are very precisely detectable.

The device and probe are preferably suitable for use in adults and adolescents (>14 years old). Their dimensions are particularly preferably designed for a craniometrical anatomy that corresponds to the majority of white Caucasian/Asian people at the time of filing the present patent, in particular a defined ear canal with a preferably minimum length of 12 mm and a preferably maximum length of 25 mm.

Further preferred embodiments of the present invention are the subject of the following description and/or the sub-claims.

According to a further preferred embodiment of the present invention, the radiation source is arranged or designed on a front side of a substrate for emitting radiation in the direction of a tympanic membrane, in particular in the direction of prolongation of the insertion portion and the radiation detector is particularly preferably also arranged or formed on the front side of the substrate for detecting radiation from the direction of the tympanic membrane, in particular from the direction of prolongation of the insertion portion. The substrate is particularly preferably a printed circuit board (PCB), wherein the PCB is preferably connectable or connected to the evaluation portion by means of lines, in particular flexible lines, such as cables, for transmitting the detected signals and/or data. Preferably, a radiation barrier is formed between the radiation source and the radiation detector, wherein the radiation source is formed as at least two light-emitting means, in particular a first LED or OLED and a second LED or OLED, for emitting radiation of different wavelengths. In the context of the present invention, a radiation barrier is understood to be a device which prevents direct illumination of the radiation detector with radiation from the radiation source. The radiation barrier is particularly preferably formed as a wall-like device or elevation between the radiation source and the radiation detector. Preferably, the radiation barrier consists of silicon, for example. This embodiment is advantageous because the radiation emitted by the radiation source is very well reflected by the tympanic membrane and the structures behind it, so that the vital parameters can be detected very accurately.

According to a further preferred embodiment of the present invention, the elongated insertion portion is designed to position the optical sensor device at a distance of equal to or less than 7 mm from the tympanic membrane, in particular from the tympanic membrane of a defined ear canal, in particular at a distance between 7 mm and 3 mm from the tympanic membrane. In addition or as an alternative, the elongated insertion portion can comprise a length between 12 mm and 25 mm, in particular between 14 mm and 23 mm and most preferably between 16 mm and 21 mm and most preferably 18 mm or substantially 18 mm or approximately 18 mm or exactly 18 mm. Both embodiments or definitions of the insertion portion are advantageous because they advantageously specify the distance of the optical sensor device from the tympanic membrane.

According to a further preferred embodiment of the present invention, the radiation source is arranged or embodied on an upper side of a substrate for emitting radiation in the direction of an ear canal wall, in particular in the radial direction of the insertion portion, and the radiation detector is preferably also arranged or formed on the upper side of the substrate for detecting radiation from the direction of the ear canal wall, in particular from the radial direction of the insertion portion. The radiation source and the radiation detector are preferably oriented in the same direction, in particular such that radiation emitted by the radiation source is detectable by the radiation detector as a result of reflection. The substrate is particularly preferably a printed circuit board (PCB), wherein the PCB is preferably connectable or connected to the evaluation portion by means of lines, in particular flexible lines, such as cables, for transmitting the detected signals and/or data. A radiation barrier is preferably formed between the radiation source and the radiation detector. The radiation barrier is particularly preferably formed as a wall-like device or elevation between the radiation source and the radiation detector. Preferably, the radiation barrier consists of silicon, for example. The radiation source is preferably formed as at least two light-emitting means, in particular a first LED or OLED and a second LED or OLED, for emitting radiation of different wavelengths. This embodiment is advantageous because the radiation source and the radiation detector are aligned very close to the tympanic membrane in the direction of the ear canal wall, whereby very precise vital parameters are detectable.

According to a further preferred embodiment of the present invention, the elongated insertion portion is designed to position the optical sensor device in a portion of the auditory canal surrounded by bone and/or at a distance equal to or less than 12 mm from the tympanic membrane, in particular the tympanic membrane of a defined auditory canal, and in particular at a distance between 7 mm and 3 mm from the tympanic membrane. In addition or as an alternative, the elongate insertion portion comprises a length between 12 mm and 25 mm, in particular between 14 mm and 23 mm and particularly preferably between 16 mm and 21 mm and most preferably 18 mm or substantially 18 mm or approximately 18 mm or exactly 18 mm. Both embodiments or definitions of the insertion portion are advantageous because they favorably predetermine the positioning of the optical sensor device very close to the tympanic membrane.

According to a further preferred embodiment of the present invention, a light-emitting means or a first light-emitting means of the radiation source is adapted to emit radiation having wavelengths in the range of 860 nm and 900 nm, in particular in the range of 875 nm and 885 nm and preferably of 880 nm. In addition or alternatively, according to a further preferred embodiment of the present invention, radiation having wavelengths in the range of 620 nm and 700 nm, in particular in the range of 655 nm and 665 nm and preferably of 660 nm is emitable by a light-emitting means respectively another light-emitting means or a further light-emitting means of the radiation source. In addition or in the alternative, according to a further preferred embodiment of the present invention, the radiation source comprises a light-emitting means, respectively a further light-emitting means, respectively a third light-emitting means, wherein radiation in the range of 480 nm and 580 nm and preferably in a range of 510 nm and 545 nm and particularly preferably in a range of 520-535 nm is emittable by the third light-emitting means. The radiation detector is configured particularly preferably for detecting radiation from the light-emitting means, from the other light-emitting means and from the third light-emitting means. This embodiment is advantageous because a defined vital parameter, in particular the oxygen saturation, can be detected by means of the radiation of the first light-emitting means and/or the radiation of the second light-emitting means. The embodiment is also advantageous because, in addition or alternatively, the pulse rate can be detected by means of the light-emitting means or the third light-emitting means, respectively.

According to a further preferred embodiment of the present invention, the radiation source and/or the radiation detector are enclosed, in particular embedded, by a material that is transparent to the radiation of the radiation source and elastically deformable, in particular a polymer material. This embodiment is advantageous because the elastically deformable material protects the very vulnerable components of the ear canal. Furthermore, the transparent material makes it possible to detect the vital parameter(s) optically.

According to a further preferred embodiment of the present invention, the substrate is mostly enclosed by an elastically deformable material, in particular a polymer material, wherein the deformable material forms at least one recess, wherein the radiation source and/or the radiation detector are arranged in the recess. The embodiment is advantageous because the requirement of transparency of the deformable material is not given, whereby the deformable material can be selected more cost-effectively, in particular if the deformable material would otherwise have to be transparent for several wavelength ranges.

In accordance with a further preferred embodiment of the present invention, a surface of the radiation source, via which the radiation of the radiation source can be emitted, is arranged at a first distance from the substrate, and wherein an outer surface of the deformable material, at least in the area surrounding the recess, comprises a second distance from the substrate, the second distance being either the same or greater than the first distance. This embodiment is advantageous because it defines the minimum distance between the sensor device and the ear canal surface.

In a preferred embodiment of the present invention, the elongate insertion portion insertable into the ear canal forms an inner end in its longitudinal direction and the elongate insertion portion comprises or forms one or at least one sealing element for closing the ear canal, the temperature sensor being arranged or formed between the inner end and the sealing element. This embodiment is advantageous because the temperature sensor can be arranged in an area in which the core body temperature is detectable.

In accordance with a preferred embodiment of the present invention, the arrangement of the temperature sensor for determining the core body temperature is configured in such a way that the temperature sensor can be arranged at a distance of 9 mm and 2 mm or at a distance of less than 9 mm, in particular less than 7 mm or between 7 mm and 2 mm, and/or at a distance of at least 0.1 mm from the tympanic membrane, and wherein the temperature sensor is arranged to determine the air temperature of a volume of air present between the sealing element and the inner end. This embodiment is advantageous because the sealing element isolates an area of the ear canal from the surrounding area and the air present in this area can thus be heated very quickly to the level of the core body temperature. This makes it possible to take very accurate temperature measurements within a short time.

In a preferred embodiment of the present invention, the evaluation portion and the temperature sensor are connected to one another in terms of signals at least temporarily, in particular in the coupled state. The temperature sensor is preferably a sensor different from an optical sensor. The temperature sensor is particularly preferably a thermistor, wherein the temperature sensor is preferably arranged or formed on a rear side of the substrate respectively PCB. The rear side is preferably parallel or preferably partially parallel to the upper side. This solution is advantageous because several functional groups can be arranged very close to the tympanic membrane respectively very deep in the ear canal, in particular in the inner part of the ear canal surrounded by bone, using a very small PCB.

The above-mentioned task is also solved by a method according to claim 12 for measuring vital parameters of a human being. The method according to the invention preferably comprises at least the steps of: providing a device for detecting at least two vital parameters, in particular a device according to the invention, introducing the elongated insertion portion into an auditory canal of a human, positioning the optical sensor device at a distance of 7 mm or less than 7 mm from the tympanic membrane, positioning the temperature sensor at a distance of 16 mm or less than 16 mm from the tympanic membrane, isolating a volume fraction of the auditory canal that contains air by means of a sealing element of the elongated insertion component from the surrounding area, generating temperature data, in particular core temperature data, the temperature data representing the temperature of the air located in the isolated volume fraction after a defined minimum period after the volume portion has been isolated from the surrounding area, the temperature data being generated by the temperature sensor or being derived from analog sensor signals of the temperature sensor after the temperature sensor has detected the temperature of the air located in the isolated volume portion, generating vital parameter data, the vital parameter data being generated by the optical sensor device or derived from analog sensor signal data of the optical sensor device, wherein the vital parameter data represents at least one vital parameter of the human being, wherein the optical sensor device has detected the at least one vital parameter, operating a display device in dependence on the temperature data and/or the vital parameter data. This solution is advantageous because a vital parameter or a variety of vital parameters can be detected very precisely by placing the sensor device in close proximity to the tympanic membrane.

In a further preferred embodiment of the present invention, the optical sensor device is positioned at a distance of 6 mm or less from the tympanic membrane, and the temperature sensor is preferably positioned at a distance of 16 mm or less from the tympanic membrane, and the evaluation portion and the elongated insertion portion are preferably connected to one another via a plug connection for establishing a data connection, in particular a data and energy connection, wherein the optical sensor device transmits the vital parameter data or vital parameter signals to the evaluation portion and wherein the temperature sensor transmits the temperature data or temperature signals to the evaluation portion. In addition or as an alternative, the device for detecting at least two vital parameters, in particular oxygen saturation and pulse rate, can be connected to a cover for completely covering the ear and preferably for at least partially and preferably completely covering of the mastoid, wherein the covering comprises the indicating means, in particular a display, wherein the indicating means is controlled in dependence on the vital parameter data and/or core temperature data. In addition or as an alternative, the evaluation portion can comprise a communication interface for wireless data exchange, wherein a data connection for transmitting the vital parameter data and/or the temperature data or for transmitting evaluation data generated by the evaluation portion is established between the communication interface and a mobile terminal, wherein the evaluation data are generated from the vital parameter data and/or the temperature data.

Some or all of the representations of the figures described below are preferably to be regarded as design drawings, i.e. the dimensions, proportions, functional relationships and/or arrangements resulting from the figures correspond preferably exactly or preferably essentially to those of the device(s), product, system, respectively, according to the invention, respectively, visualize(s) essentially or exactly steps of the method(s) according to the invention.

In which:

FIG. 1a shows a schematic view of an exemplary device according to the invention for insertion into the ear canal of a human;

FIG. 1b shows a schematic and enlarged view of the sensor device merely sketched in FIG. 1a;

FIG. 1c shows a schematic view of the exemplary device according to the invention in accordance with FIG. 1a inserted into the ear canal of a human;

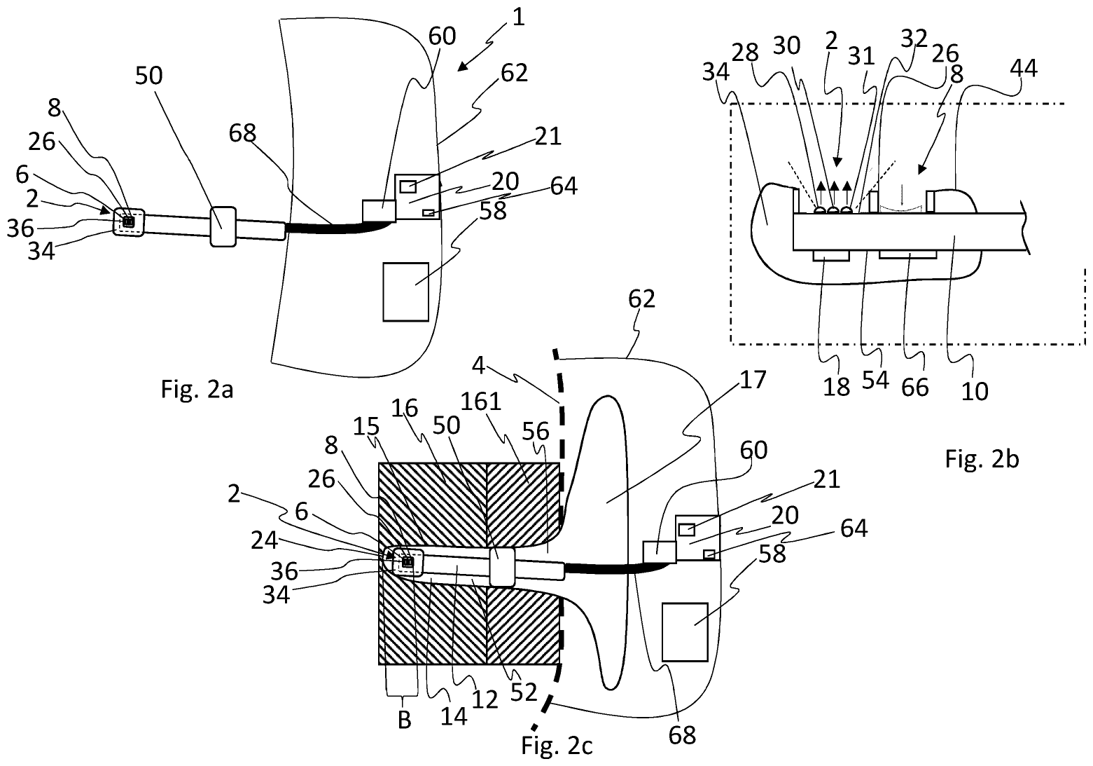

FIG. 2a a schematic view of a further exemplary device according to the invention for insertion into the ear canal of a human;

FIG. 2b a schematic and enlarged view of the sensor device merely sketched in FIG. 2a;

FIG. 2c a schematic view of the exemplary device according to the invention as shown in FIG. 2a inserted into the ear canal of a human; and

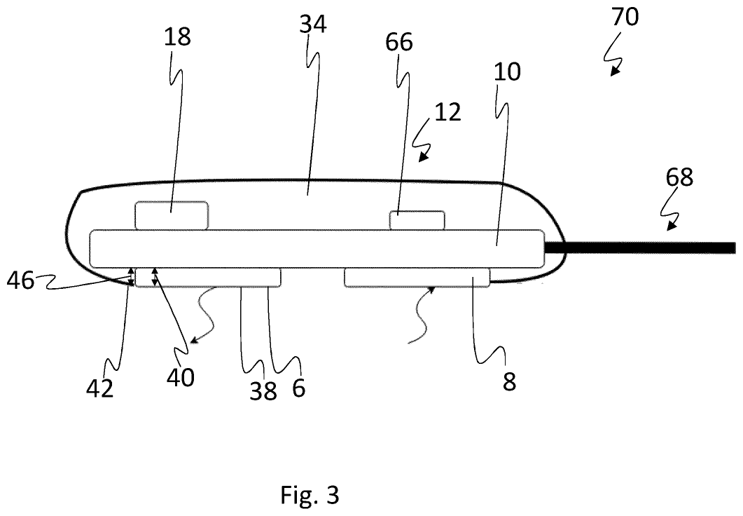

FIG. 3 shows a schematic view of an alternative sensor head of a device according to the invention.

FIG. 1a shows an example of a device according to the invention 1. The device according to the invention 1 is preferably formed for detecting one or more vital parameters. The core body temperature, the oxygen saturation of the blood or the pulse frequency, for example, are regarded as vital parameters. The device 1 can thus be designated as a device for detecting vital parameters or alternatively as a pulse rate detection device or as an oxygen saturation detection device or as a core body temperature detection device. Preferably, the device 1 comprises a plurality of parts that can be releasably coupled to one another, namely an elongated insertion portion 12 and a cover 62. The elongated insertion portion 12 is preferably connectable to the cover 62 by means of a coupling device 60, in particular a plug connection, via which signals and/or data and/or electricity are preferably transmissible. Alternatively, however, it is also possible that the elongated insertion portion 12 and the cover are non-detachably connected to one another.

The elongated insertion portion 12 is designed for insertion into the ear canal of a living being 4 (see FIG. 1c), in particular a human being, and comprises at least one sensor device, in particular an optical sensor device 2 and/or a temperature sensor 18. The optical sensor device 2 is preferably arranged or formed on a front side 22 (see FIG. 1b) of the elongated insertion portion 12. The front side 22 is preferably aligned essentially orthogonally to the longitudinal direction L of the elongated insertion portion 12. Preferably, the front side 22 forms at least part, or a majority or all, of one end 48 (see FIG. 1b) of the elongate insertion portion 12. The end formed by the front side 22 can also be referred to as the inner end 48, since it forms or represents the portion of the elongate insertion portion 12 that can be inserted the furthest in the longitudinal direction L of the elongate insertion portion 12 into the auditory canal 14 of the ear 17 (see FIG. 1c) of the living being 4 (FIG. 1c) The optical sensor device 2 preferably comprises a radiation source 6 and a radiation detector 8 for detecting radiation 7 that has been emitted by the radiation source 6. The radiation source 6 and the radiation detector 8 are particularly preferably arranged next to one another, in particular at a distance of less than 5 mm or preferably less than 4 mm or particularly preferably less than 3 mm from one another. It is possible here that a radiation barrier 26 is provided or formed between the radiation source 6 and the radiation detector 8. The radiation barrier can, for example, consist of the same material as the substrate 10 (see FIG. 1b). However, the radiation barrier 28 is not permeable, at least for radiation from the radiation source 6.

The optical sensor device 2 preferably comprises at least one light-emitting means 28 and preferably two or more than two light-emitting means 28 and 30 and particularly preferably three or more than three light-emitting means 28, 30 and 31 (see FIG. 1b). Preferably, the first light-emitting means 28 of the radiation source 6 can emit radiation with wavelengths in the range of 860 nm and 900 nm, in particular in the range of 875 nm and 885 nm and preferably of 880 nm. Another respectively second light-emitting means 30 of the radiation source 6 is preferably designed to emit radiation with wavelengths in the range of 620 nm and 700 nm, in particular in the range of 655 nm and 665 nm and preferably of 660 nm. Preferably, the radiation source 6 can comprise at least a third light-emitting means 31, wherein the third light-emitting means 31 is adapted to emit radiation in the range of 480 nm and 580 nm and preferably in a range of 510 nm and 545 nm and particularly preferably in a range of 520-535 nm. It is of course possible that, depending on the required vital parameters, the light-emitting means designated as the third light-emitting means 31 is understood as the first light-emitting means or as the second light-emitting means. Similarly, it may be the case for the second light-emitting means 30 that it can alternatively be understood as the first or third light-emitting means. The first light-emitting means 29 can thus alternatively be understood as the second or third light-emitting means.

The radiation detector 8 for detecting radiation 7 from the radiation source 6 is thus configured to detect radiation from the first light-emitting means 28 and from the second light-emitting means 30 and from the third light-emitting means 31 and to convert it into data or analog signals.

The temperature sensor 18 is preferably arranged or formed on a surface of the elongated insertion portion 12 that is inclined with respect to the front side 22 (see FIG. 1b). Furthermore, a capacitor 66, in particular for supplying the radiation source 6 and/or the radiation detector 8 and/or the temperature sensor 18 with electrical energy, can be arranged or formed on the substrate.

Furthermore, the elongated insertion portion 12 preferably comprises a sealing element 50 for sealing an ear canal 14 (see FIG. 1c). The sealing element 50 is preferably arranged on the elongated insertion portion 12 in a permanent or exchangeable manner. It is particularly preferred that the position of the sealing element 50 can be adjusted or displaced in the longitudinal direction L of the elongated insertion portion 12. Alternatively, however, it is also possible for the sealing element 50 to be arranged fixedly (but preferably replaceably) at a defined position.

The reference sign 68 designates a connecting element which is preferably connected by means of a coupling device 60, in particular a detachable plug connection or a detachable magnetic connection, to the evaluation portion 20, which preferably has at least one microprocessor 21 for processing the vital parameter data and/or for operating the sensor device 2 and/or the temperature sensor 18.

Signals and/or data and/or electrical energy can be conducted between the sensor device 2 and/or the temperature sensor 18 and the evaluation portion 20 by means of one or more conducting elements (not shown). The conducting element or elements preferably run inside the elongated insertion portion 20 and inside the connecting element 68. The elongated insertion portion 20 and the connecting element 68 can alternatively be designed as a continuous device or as a device with a common housing or enclosure, in particular as an elongated insertion portion 20 with an outer connecting section. The reference sign 58 designates an indicating device, in particular for displaying information, in particular vital parameter results or changes in vital parameters or operating instructions. Furthermore, input means (not shown), in particular buttons and switches, can also be provided for entering control commands. The display device 58 is preferably formed as a touch screen and thus, in addition to the optical output of information, also enables the selection or activation of functions. Furthermore, the device 1 can comprise an acoustic output device (not shown) for the acoustic output of information, in particular vital parameter results or changes in vital parameters or operating instructions. Furthermore, the evaluation portion 20 preferably comprises a communication means or a communication device 64, respectively, in particular a communication device for wireless data exchange, in particular for data exchange by radio transmission, such as NFC or Bluetooth or WLAN or 3G, 4G, 5G or 6G, or is connected to such a communication device 64.

FIG. 1b shows an enlarged representation of the part of the device 1 that can be arranged in the area of the tympanic membrane 24 (see FIG. 1c). It can be seen that the elastically deformable material 34 preferably comprises or forms a recess, wherein the radiation 7 that is emitted by the light-emitting means 28, 30, 31, can be introduced into the living creature 4 via the tympanic membrane 24 and reflected radiation 7 can reach the radiation detector 8 without the radiation having to penetrate non-biological material. The reference sign 36 characterizes a recess, i.e. an area in the beam path of the radiation 7 that is not filled by the elastic material 34. Alternatively, the elastically deformable material 34 can completely encase the sensor device 2, wherein in this case the elastically deformable material would have to be transparent for the radiation 7 emitted by the at least one light-emitting means and preferably by the light-emitting means.

FIG. 1c schematically shows a device 1 according to the invention, wherein the elongated insertion portion 12 is inserted into the ear canal 14 of a living being 4. The elastically deformable portion 34 contacts the auditory canal wall 15 and prevents, for example, sharp-edged portions of the elongated portion 12 from coming into contact with the very sensitive auditory canal wall 15.

In the arrangement shown, the sensor device 2 is arranged at a distance A of less than 7 mm from the tympanic membrane 24.

The sealing element 50 is preferably arranged in such a way that it is positioned in a part 161 of the auditory canal 14 that is surrounded by tissue. Alternatively, the sealing element 50 can also be arranged in such a way that it is positioned in a part 16 of the auditory canal 14 surrounded by bone. As a result of the positioning of the sealing element 50, a volume of air 52, which is located between the sealing element 50 and the tympanic membrane 24, is isolated from the surrounding area 56.

FIGS. 2a-2c show a further example of the device 1 according to the invention. The device 1 shown in FIGS. 2a-2c is essentially the same as the device 1 shown in FIGS. 1a-1 c, wherein only the arrangement of the sensor device 2 and/or the temperature sensor 18 is different, whereby the same reference signs are used for the same components and the above explanations apply by analogy.

It can be seen, for example, in FIG. 2b that the radiation source 6 is arranged or formed on an upper side 32 of a substrate 10 for emitting radiation 7 in the direction of an ear canal wall 15 (see FIG. 2c), in particular in the radial direction of the insertion portion 12. The radiation detector 8 is also arranged or formed on the upper side 32 of the substrate 10 for detecting radiation 7 from the direction of the ear canal wall 15 (see FIG. 2c), in particular from the radial direction of the insertion portion 12. The substrate 10 is preferably designed as a PCB and, between the radiation source 6 and the radiation detector 8, a radiation barrier 26 is particularly preferably formed. The radiation source 6 can be formed as at least two light-emitting means 28, 30, in particular a first LED or OLED and a second LED or OLED, for emitting radiation 7 of different wavelengths. The elongated insertion portion 12 is preferably designed so that the optical sensor device 2 can be positioned in a portion 16 of the auditory canal 14 surrounded by bone and/or at a distance (B) equal to or less than 12 mm from the tympanic membrane 24 (see FIG. 2c), in particular to the tympanic membrane 24 of a defined auditory canal 14, in particular at a distance between 7 mm and 3 mm to the tympanic membrane 24 (see FIG. 2c).

FIG. 3 shows a further example of a sensor head 70. The sensor head 70 is formed by the elongated insertion portion 12 and at least one portion of the connecting element 68. The reference sign 38 characterizes a surface spaced from the substrate 10, via which the radiation 7 can be emitted. The surface 38 is spaced at a distance 40 from the nearest substrate surface portion. The reference sign 34 designates an elastic material which is provided for protecting the auditory canal 14 (see FIG. 1c or FIG. 2c). The elastic material comprises a region which is adjacent to the radiation source 6, in particular directly adjacent, and is spaced at a distance 42 from the nearest substrate surface portion. The distance 42 is preferably equal to the distance 40 or the distance 42 is greater than the distance 40.

The present invention thus generally relates to a device 1 for detecting at least two vital parameters, wherein this device 1 comprises an optical sensor device 2, an elongated insertion portion 12 and an evaluation portion 20. The optical sensor device 2 is preferably used for detecting at least one vital parameter of a living being 4, in particular a human being, wherein the optical sensor device 2 comprises a radiation source 6 for emitting radiation 7 and a radiation detector 8 for detecting radiation 7, wherein the radiation source 6 and the radiation detector 8 are particularly preferably arranged next to one another. The elongated insertion portion 12 is preferably used for insertion into an ear canal 14 and for positioning the optical sensor device 2 in a portion 16 of the ear canal 14 surrounded by bone. The optical sensor device 2 is preferably arranged on the elongated insertion portion 12 or is part thereof. A temperature sensor 18 for detecting the core body temperature is preferably arranged on the elongated insertion portion 12 or is part of it. The evaluation portion 20 is preferably coupled to the insertion portion 12, in particular detachably coupled, the evaluation portion 20 and the optical sensor device 2 being connected to one another in terms of signaling at least temporarily, in particular in a coupled state. Furthermore, it can be seen from FIG. 2b that the temperature sensor 18 and/or the capacitor 66 is/are arranged or formed on a rear side 54 facing away from the upper side 32. However, it is alternatively possible that the temperature sensor 18 and/or the capacitor 66 is arranged or formed on the same side, i.e. for example on the upper side 32, on which the sensor device 2 is also arranged or formed.

### LIST OF REFERENCE SIGNS

1 device for detecting at least one vital parameter and preferably at least two vital parameters
2 optical sensor device
4 living being
6 radiation source
7 radiation
8 radiation detector
10 substrate
12 elongated insertion portion
14 auditory canal
15 auditory canal wall
16 portion surrounded by bone
17 ear
18 temperature sensor
20 evaluation portion
22 front side
24 tympanic membrane
26 radiation barrier
28 first light-emitting means
30 second light-emitting means
31 third light-emitting means
32 upper side
34 elastically deformable material
36 recess
38 surface
40 first distance
42 outer surface
44 surrounding area
46 second distance
48 inner end
50 sealing element
52 air volume
54 rear side
56 surrounding area
58 display device
60 plug connection
62 cover
64 communication interface
66 capacitor
68 connecting element
70 sensor head
161 portion surrounded by tissue
A distance between the tympanic membrane and the sensor device arranged on the front side
B distance between the tympanic membrane and the sensor device arranged on the top side
L longitudinal direction of the elongated insertion portion

1. A device (1) for detecting at least two vital parameters, the device comprising
an optical sensor device (2) for detecting at least one vital parameter of a living being (4), wherein the optical sensor device (2) comprises a radiation source (6) for emitting radiation (7) and a radiation detector (8) for detecting radiation (7), wherein the radiation source (6) and the radiation detector (8) are arranged next to one another,
an elongated insertion portion (12) for insertion into an ear canal (14) and for positioning the optical sensor device (2) in a portion (16) of an ear canal (14) surrounded by bone,
wherein the optical sensor device (2) is arranged on the elongated insertion portion (12) or is a component thereof and wherein a temperature sensor (18) for detecting a core body temperature is arranged on the elongated insertion portion (12) or is a component thereof,
an evaluation portion (20) coupled to the insertion portion (12), wherein the evaluation portion (20) and the optical sensor device (2) are at least temporarily, in particular in a coupled state, connected to one another in terms of signaling,
wherein the radiation source (6) is arranged or formed on a front side (22) of a substrate (10) for emitting radiation (7) in the direction of a tympanic membrane (24) in the direction of prolongation of the insertion portion (12), and
the radiation detector (8) is arranged or formed on the front side (22) of the substrate (10) for detecting radiation (7) from the direction of the tympanic membrane (24) in the direction of prolongation of the insertion portion (12),
wherein the substrate (10) is a PCB and
wherein a radiation barrier (26) is formed between the radiation source (6) and the radiation detector (8),
wherein the radiation source (6) is formed as at least two light-emitting means (28, 30), for emitting radiation (7) of different wavelengths,
wherein the elongated insertion portion (12) insertable into the ear canal (14) forms an inner end (48) in its longitudinal direction (L) and wherein the elongated insertion portion (12) comprises or forms one or at least one sealing element (50) for closing the auditory canal (14), wherein the temperature sensor (18) is arranged or formed between the inner end (48) and the sealing element (50).

2. The device according to claim 1,
characterized in that
the elongated insertion portion (12) is designed to position the optical sensor device (2) at a distance of equal to or less than 7 mm from the tympanic membrane (24),
and/or
the elongated insertion portion (12) comprises a length between 12 mm and 25 mm.

3. The device according to claim 1,
characterized in that
the radiation source (6) is arranged or formed on an upper side (32) of a substrate (10) for emitting radiation (7) in the direction of an ear canal wall (15), and
the radiation detector (8) is arranged or formed on the upper side (32) of the substrate (10) for detecting radiation (7) from the direction of the auditory canal wall (15), in particular from the radial direction of the insertion portion (12),
wherein the substrate (10) is a PCB and
wherein a radiation barrier (26) is formed between the radiation source (6) and the radiation detector (8),
wherein the radiation source (6) is formed as at least two light-emitting means (28, 30), in particular a first LED or OLED and a second LED or OLED, for emitting radiation (7) of different wavelengths.

4. The device according to claim 3,
characterized in that
the elongated insertion portion (12) is designed to position the optical sensor device (2) in a portion (16) of the auditory canal (14) surrounded by bone and/or at a distance (B) equal to or less than 12 mm from the tympanic membrane (24), in particular from the tympanic membrane (24) of a defined auditory canal (14), in particular at a distance between 7 mm and 3 mm from the tympanic membrane (24)
and/or
the elongate insertion portion (12) comprises a length between 12 mm and 25 mm.

5. The device according to claim 1,
characterized in that
radiation with wavelengths in the range of 860 nm and 900 nm is emitted by a light-emitting means (28) of the radiation source (6)
and
wherein another light-emitting means (30) of the radiation source (6) is adapted to emit radiation having wavelengths in the range of 620 nm and 700 nm.

6. The device according to claim 5,
characterized in that
the radiation source (6) comprises at least a third light-emitting means (31), wherein radiation in the range of 480 nm and 580 nm is emittable by the third light-emitting means (31),
wherein the radiation detector (8) is configured to detect radiation (7) from the light-emitting means (28), the other light-emitting means (30) and the third light-emitting means (31).

7. The device according to claim 1, characterized in that
the radiation source (6) and/or the radiation detector (8) are enclosed by a material (34) which is elastically deformable and transparent to the radiation (7) of the radiation source (6).

8. The device according to claim 1, characterized in that
the substrate (10) is mostly enclosed by an elastically deformable material (34), wherein the elastically deformable material (34) forms at least one recess (36), wherein the radiation source (6) and/or the radiation detector (8) are arranged in the recess (36).

9. The device according to claim 8,
characterized in that
a surface (38) of the radiation source (6), via which the radiation (7) of the radiation source (6) can be emitted, is arranged at a first distance (40) from the substrate (10), and wherein an outer surface (42) of the elastically deformable material (34) comprises a second distance (46) from the substrate (10) at least in the area (44) surrounding the recess (36), wherein the second distance (46) is either the same size as or larger than the first distance (40).

10. The device according to claim 1,
characterized in that
the arrangement of the temperature sensor (18) for determining the core body temperature is configured in such a way that the temperature sensor (18) can be arranged at a distance of between 9 mm and 0.1 mm from the tympanic membrane (18), and wherein the temperature sensor (18) is set up to determine the air temperature of a volume of air (52) present between the sealing element (50) and the inner end (48).

11. The device according to claim 10, characterized in that the evaluation portion (20) and the temperature sensor (18) are connected to one another in terms of signals at least temporarily, wherein the temperature sensor (18) is a sensor other than an optical sensor, wherein the temperature sensor (18) is a thermistor.

12. A method for measuring vital parameters of a human (4), comprising:
providing a device (1) for detecting at least two vital parameters according to claim 1,
inserting an elongated insertion portion (12) into an ear canal (14) of a human (4),
positioning the optical sensor device (2) at a distance of 7 mm or less from a tympanic membrane (24),
positioning a temperature sensor (18) at a distance of 16 mm or less from the tympanic membrane (24),
isolating a volume portion (52) of the ear canal (14) containing air from a surrounding area (56) by means of a sealing element (50) of the elongated insertion portion (12),
generating temperature data, wherein the temperature data represent a temperature of the air located in the isolated volume portion (52) after a defined minimum period of time after the volume portion (52) has been isolated from the surrounding area (56), wherein the temperature data are generated by the temperature sensor (18) or are derived from analog sensor signals of the temperature sensor (18) after the temperature sensor (18) has detected the temperature of the air in the defined volume fraction,
generating vital parameter data, wherein the vital parameter data are generated by the optical sensor device (2) or are derived from analog sensor signal data of the optical sensor device (2), wherein the vital parameter data represent at least one vital parameter of the human (4), wherein the optical sensor device (2) has detected the at least one vital parameter, and
operating a display device (58) in dependence on the temperature data and/or the vital parameter data.

13. The method according to claim 12,
characterized in that
the optical sensor device (2) is positioned at a distance of 6 mm or less from the tympanic membrane (24), and
the temperature sensor (18) is positioned at a distance of 16 mm or less from the tympanic membrane (24)
and
wherein the evaluation portion (20) and the elongated insertion portion (12) are connected to one another via a plug connection (60) for establishing a data connection, in particular a data and energy connection, wherein the optical sensor transmits the vital parameter data or vital parameter signals to the evaluation portion (20) and wherein the temperature sensor (18) transmits the temperature data or temperature signals to the evaluation portion (20)
and
wherein the device (1) for detecting at least two vital parameters is connected to a cover (62) for completely covering the ear (17), wherein the cover (62) comprises the display device (58), wherein the display device (58) is controlled in dependence on the vital parameter data and/or core temperature data
and
wherein the evaluation portion (20) comprises a communication interface (64) for wireless data exchange, wherein a data connection (68) for transmitting the vital parameter data and/or the temperature data or for transmitting evaluation data generated by the evaluation portion (20), wherein the evaluation data are generated from the vital parameter data and/or the temperature data.

14. The device of claim 1, wherein the at least two light-emitting means (28, 30) include a first LED or LED and a second LED or OLED.

15. The device of claim 2, wherein the elongated insertion portion (12) is designed to position the optical sensor device (2) at the distance between 7 mm and 3 mm from the tympanic membrane (24).

16. The device of claim 5, wherein radiation is emitted with wavelengths in the range of 875 nm and 885 nm by the light-emitting means (28) of the radiation source (6).

17. The device of claim 5, wherein the another light-emitting source (30) of the radiation source (6) is adapted to emit radiation having wavelengths in the range of 655 nm and 665 nm.

18. The device of claim 6, wherein radiation in the range of 520 nm and 535 nm is emittable by the third light-emitting means (31).

19. The device of claim 11, wherein the temperature sensor is arranged or formed on a rear side (54) of the substrate (10).

20. The method of claim 13, wherein the two vital parameters for detecting by device (1) are an oxygen saturation and a pulse rate.
